(19) United States
(12) Patent Application Publication
(10) Pub. No.: US 2023/0172832 A1
(43) Pub. Date: Jun. 8, 2023

(54) PERSONAL CARE COMPOSITION COMPRISING MANNOSE OLGOSACCHARIDES

(71) Applicant: CARGILL, INCORPORATED, Wayzata, MN (US)

(72) Inventors: Nabila BELHAJ, Saint Cloud (FR); Dominique Karine CLIO, Oplinter (BE); Jean-Noel OLLAGNIER, Evere (BE); Bruno Frederic STENGEL, Brussels (BE)

(73) Assignee: CARGILL, INCORPORATED, Wayzata, MN (US)

(21) Appl. No.: 17/906,099

(22) PCT Filed: Mar. 9, 2021
(86) PCT No.: PCT/US2021/021494

§ 371 Date: Sep. 12, 2022

Related U.S. Application Data
(60) Provisional application No. 62/989,089, filed on Mar. 13, 2020.
(60) Provisional application No. 63/034,457, filed on Jun. 4, 2020.

(51) Int. Cl. A61K 8/60; A61Q 19/08

(52) U.S. Cl. CPC A61K 8/60; A61Q 19/08

(57) ABSTRACT

The present invention relates to a highly purified composition comprising mannose oligosaccharide and a process for making such. The present invention further relates to the use of a highly purified composition comprising mannose oligosaccharide to stimulate the production of collagen I, collagen III, and/or Fibrillin 1 in the skin of a mammal.

### CROSS-REFERENCE TO RELATED APPLICATIONS

This application claims the benefit of U.S. Provisional Patent Application No. 62/989,089, filed Mar. 13, 2020, and U.S. Provisional Patent Application No. 63/034,457, filed Jun. 4, 2020, each of which is hereby incorporated by reference in its entirety.

### FIELD OF THE INVENTION

The present invention relates to a composition comprising mannose oligosaccharides and a process for making such a composition. The present invention further relates to personal care products comprising the composition of the present invention. The present invention further relates to use of the disclosed compositions for the treatment of skin to prevent ageing or to improve skin structure.

### BACKGROUND OF THE INVENTION

There is an existing demand for cosmetic ingredients useful for improving overall health of the skin. As we age, it is known that the structure of the skin changes over time. Loss of elasticity, loss of resistance to dehydration, changes in coloration, and increase in wrinkles are some of the changes experienced as we age.

There is a continuing effort to understand and affect these common changes to the health of the skin. Numerous products are evaluated and commercialized every year to this end. A need still exists to identify cost effective and naturally sourced products to address this ubiquitous interest for all humans.

Mannose oligosaccharides compositions are known to improve overall health, in particular gut health but nothing has been known about their effect on skin health. Existing mannose oligosaccharides (or manno-oligosaccharide, or MOS) compositions currently on the market are all extracts, such as yeast extracts, mostly yeast cell wall extracts, green coffee bean extracts, konjac extracts and so on. They all present the disadvantage of not being highly pure compositions. Rather they are inconsistent compositions, particularly true for yeast cell wall extracts, and consequently their action in animals is not predictable and their effects fluctuate. In addition, these extracts typically have poor or variable solubility in water. As a consequence, their application in food and cosmetic applications is limited.

### SUMMARY OF THE INVENTION

In a first aspect, the present invention relates to the surprising observation by the inventors that a highly purified a composition comprising mannose oligosaccharide is effective in modifying the health of aging skin cells. This is a completely novel and unexpected effect.

An additional aspect of the present invention is a mannose oligosaccharide composition, characterized in that said composition is highly purified.

An additional aspect of the present invention is a mannose oligosaccharide composition, characterized in that has a DPn of >50%, a dry substance content of >65%, a Gardner color value of less than 3.0 and/or a conductivity value of a 50% by weight solution in water of <500 microsimons/cm.

In a further aspect, the present invention relates to a cosmetic, personal care or pharmaceutical product comprising the MOS composition of the present invention.

In a further aspect, the present invention relates the use the MOS composition of the present invention as a cosmetic active agent preferably as an anti-aging cosmetic active agent for stimulating the production of collagen I, collagen III, and/or Fibrillin 1.

In a further aspect, the present invention relates the use the MOS composition of the present invention to promote or assist in healing of wounds to the skin.

In a further aspect, the present invention relates to a process for making a highly purified composition comprising mannose oligosaccharide characterized in that it comprises the steps of:
a) Polycondensing mannose by passing the mannose through a microreactor at a temperature of from 180° C. to 240° C., preferably from 195° C. to 240° C. in the presence of an acidifying catalyst to produce a manno-oligosaccharide containing composition,
b) Neutralising the manno-oligosaccharide containing composition from step a),
c) Decolorizing the neutralized composition of step b),
d) De-salting the of decolorized solution of step c), by electrodialysis and/or ion exchange treatment,
e) Polishing of product resulting from step d), and
f) collecting the MOS.

### BRIEF DESCRIPTION OF THE DRAWINGS

FIG. 1 shows the effect of compositions disclosed herein on epithelial thickness.

FIG. 2 shows the effect of compositions disclosed herein on Collagen I.

FIG. 3 shows the effect of compositions disclosed herein on Collagen III.

FIG. 4 shows the effect of compositions disclosed herein on Fibrillin I.

### DETAILED DESCRIPTION

The use of “a” or “an” to describe the various elements or components herein is merely for convenience and to give a general sense of the invention. This description should be read to include one or at least one, and the singular also includes the plural unless it is obvious that it is meant otherwise.

In the present description, the terms mannose oligosaccharide, manno-oligosaccharide and MOS are used interchangeably to describe the same. Mannose oligosaccharide is defined as an oligosaccharide of mannose having a DP of 3 or more. DP refers to the degree of polymerisation, i.e. the number of monomers present in the oligosaccharide. In mannose oligosaccharide, the monomer is mannose. The mannose oligosaccharide composition of the present invention comprises mannose oligosaccharides having a DP of 3 or more (also referred to as “DPn”) and is characterized in that it is also source of mannose disaccharide, further it may comprise mannose monosaccharide. HPLC analysis (ISO 10504:1998-10) may be done to determine the amount and type of the various saccharide present in the mannose oligosaccharide composition, such as DP1, DP2, DP3 and higher.

Manno-oligosaccharide compositions of the present invention have very low Gardner color values. In aspects of the invention: Gardner color value is less than 5.0, less than 3.0, between 1.0 and 5.0, or between 1.0 and 3.0; and can be described as a faintly yellow clear liquid. By way of comparison the manno-oligosaccharide composition directly after step (b) has a Gardner color value of 12-15 and can be described as espresso colored.

Gardner color is a two-dimensional scale for yellowness. The yellowness of the transparent liquid is determined by pouring the sample into a tube and comparing it to a pre-determined and known standard. The standard that the sample falls closest to then becomes the value for the liquid. Procedures for Gardner color evaluation are well known in the art and includes the standard Test ASTM D1544 Method for Color of Transparent Liquids (Gardner Color Scale),” see http://www.astm.org/Standards/D1544.htm.

Manno-oligosaccharide compositions of the present invention have very low furfural and hydroxymethylfurfural impurity levels. Furfural and hydroxymethylfurfural are known impurities in carbohydrate products and are especially problematic for cosmetic and personal care products. Preferable manno-oligosaccharide compositions of the present invention contain less than 1 ppm of furfural or hydroxymethylfurfural. More preferably the levels of furfural and hydroxymethylfurfural are below detection limit. Presence of these impurities are well known to those in the carbohydrate arts and can be determined by procedures well known in the art including HPLC. For example, according to the following HPLC conditions:

Column e g Aminex A-7, Ca²⁺-form, 0.8*30 cm

Flow rate: 0.7 ml/min

Eluent: 0.005 M Ca (NO₃)₂*4H₂O

Temperature: 60° C.

Injection volume: 20 μl

Detection: UV-Monitor

Wavelength: 284 nm

Standards for HMF or Furfural: app. 0.02 g/250 ml

Preferably the MOS composition of the present invention has a content of DPn>50%, >55%, or >60% or >65% or >70%, or >90%. These values are percent by weight values on a dry basis. Ranges between any of these values are also envisioned by the inventors such as from 50 to 65 wt %, preferably of from 55 to 90 wt %, preferably of from 60 to 70 wt %. The content of DP2 may be about 60 wt %.

Further, the MOS composition may contain mannose in an amount of from 5 to 20 wt % on a dry basis (db), preferably from 15 to 20 wt % db.

The dry substance content, or degree of solids content, of the MOS composition can be adapted to the need of its application. The dry substance may be at least 65 wt %, preferably at least 70 wt %, more preferably at least 80 wt %, even more preferably at least 90 wt %, yet even more preferably at least 95 wt %, such as for example from 95 to 99 wt %. The dry substance of the MOS composition can be any range between 65% and 99%; such as between 70% and 95% or 80% and 99%. The MOS composition can be stored either in powder form after spray drying or in liquid form depending on the intended use. In powder form, the MOS composition of the present invention is a stable, yellowish to white, free flowing powder.

Further, the MOS composition may be characterised in that the mannose oligosaccharide comprises predominantly alpha- & beta-1,6 and alpha-1,3 type linkages. Preferably at least 50%, more preferably at least 75%, even more preferably at least 80% of the linkages are alpha- & beta-1,6 and alpha-1,3 type linkages. Methods for measuring the various types of linkages which may be present in saccharides are well known in the art, such as High-Performance Anion Exchange with Pulsed Amperometric Detection Detection (HPAE-PAD).

It has been found that the MOS composition according to the present invention has an improved effect on the known skin aging markers of i) epidermal thickness, ii) collagen I, iii) collagen II, iv) collagen III, and v) fibrillin I. In particular, highly purified MOS compositions of the present invention characterized in that DP3+ is higher than 50 wt % have an improved effect on these aging markers.

The present invention further relates to the use of, or methods of using, the MOS compositions as described herein for treating human skin.

The present invention further relates to the use of, or methods of using, the MOS compositions as described herein for improving i) epidermal thickness, ii) collagen I, iii) collagen II, iv) collagen III, or v) fibrillin I.

The present invention further relates to the use of, or methods of using, the MOS composition to prepare a cosmetic or personal care composition for treating human skin.

The present invention further relates to the use of, or methods of using, the MOS composition to prepare a cosmetic or personal care composition for promotion or acceleration of wound healing.

An advantage of using the MOS composition of the present invention is that it can readily be dissolved, it can be better handled and/or dosed and is thus very suitable for use in many applications using at least one liquid ingredient, such as for example a cream or lotion. Also, in dry mixes, the MOS composition can be easily incorporated.

Further, the present invention relates to a cosmetic product comprising the MOS composition of the present invention and further cosmetic product ingredients, particularly in products having anti-aging properties. Cosmetic products can be creams, lotions, lipsticks, makeups containing pigments and other moisturizers, and the like. Preferably the cosmetic product may comprise from 0.01 to 30 wt %, preferably from 0.01 to 20 wt %, more preferably from 1 to 15 wt %, even more preferably from 5 to 10 wt % of the present MOS composition, based on the weight of the cosmetic product. The present invention also relates to the use of the MOS composition in cosmetic products. In particular, the present invention also relates to the use of the MOS composition in cosmetic products having anti-aging properties or affects.

In one aspect, the present invention is a topical formulation comprising a manno-oligosaccharide composition described herein. As used herein, the term “topical formulation” refers to a formulation that may be applied directly to a part of the body. The term “formulation” is used herein to denote compositions of various ingredients in various weight ranges, in accordance with the present invention. Such compositions are well known in the art to the skilled person.

The formulations manufactured with the manno-oligosaccharide composition described herein are suitable for use on hair, scalp, nails and skin, for delivering cosmetic or actives to the skin or hair for providing cleansing, conditioning, moisturizing, minimizing or treating skin imperfections, reducing skin oiliness, providing fragrances to the hair or skin and the like.

“Personal care” means and comprises any hygienic, toiletry and topical care products including, without limitation, leave-on products (i.e., products that are left on the skin or keratinous substrates after application); rinse-off products (i.e., products that are washed or rinsed from the skin or keratinous substrates during or within a few minutes of application); shampoos; hair curling and hair straightening products; hair style maintaining and hair conditioning products; lotions and creams for nails, hands, feet, face, scalp and/or body; hair dye; face and body makeup; nail care products; astringents; deodorants; antiperspirants; anti-acne; antiaging; depilatories; colognes and perfumes; skin protective creams and lotions (such as sunscreens); skin and body cleansers; skin conditioners; skin toners; skin firming compositions; skin tanning and lightening compositions; liquid soaps; bar soaps; bath products; shaving products; and oral hygiene products (such as toothpastes, oral suspensions, and mouth care products). Preferably the personal care product may comprise from 0.01 to 30 wt %, preferably from 0.01 to 20 wt %, more preferably from 1 to 15 wt %, even more preferably from 5 to 10 wt % of the present MOS composition, based on the weight of the personal care product. The present invention also relates to the use of the MOS composition in personal care products. In particular, the present invention also relates to the use of the MOS composition in personal care products having anti-aging properties.

The texture of such personal care formulations is not limited and may be, without limitation, a liquid, gel, spray, emulsion (such as lotions and creams), shampoo, pomade, foam, tablet, stick (such as lip care products), makeup, suppositories, among others, any of which can be applied to the skin or hair or hale and which typically are designed to remain in contact therewith until removed, such as by rinsing with water or washing with shampoo or soap. Other forms could be gels that can be soft, stiff, or squeezable. Sprays can be non-pressurized aerosols delivered from manually pumped finger-actuated sprayers or can be pressurized aerosols such as mousse, spray, or foam forming formulation, where a chemical or gaseous propellant is used.

The personal care formulation comprising the highly purified manno-oligosacharride disclosed herein may be a cream or lotion that may optionally include sun blocking agents, moisturizers, or other active ingredients.

The invention further relates to a process for making a manno-oligosaccharide composition, said process comprises the steps of:
a. Polycondensing mannose by passing the mannose through a microreactor at a temperature of from 180° C. to 240° C., preferably from 195° C. to 240° C. in the presence of an acidifying catalyst to produce a manno-oligosaccharide containing composition,
b. Neutralising the manno-oligosaccharide containing composition from step a),
c. Decolorizing the neutralized composition of step b),
d. De-salting the of decolorized solution of step c), by electrodialysis and/or ion exchange treatment,
e. Polishing of step d), and
f. collecting the MOS.

In the methods described herein, the acts can be carried out in a specific order as recited herein. Alternatively, in any embodiment disclosed herein, specific acts may be carried out any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The mannose may be a mannose containing composition, isolated mannose, a mannose solution or a mixture of these. Preferably the mannose is an aqueous mannose solution. The concentration of the mannose in the aqueous mannose solution is not important, however, it is advantageous to have the aqueous mannose solution as concentrated as possible consistent with viscosity requirements. Therefore, it is advantageous that the aqueous mannose solution comprises from 10 to 90 wt % dry substance, preferably from 15 to 80 wt % dry substance, more preferably from 40 to 70 wt % dry substance, even more preferably from 50 to 60 wt % dry substance. Most preferably the aqueous mannose solution comprises from 75 to 90 wt % dry substance.

The amount of acidifying catalyst used is preferably in a weight ratio to mannose:acidifying catalyst 100:0.005 to 100:20, more preferably from 100:0.5 to 100:10, even more preferably from 100:1 to 100:5.

Preferably the acidifying catalyst is citric acid, sulphuric acid, and/or phosphoric acid.

The temperature maintained in the microreactor during step a) will affect the amount or degree of polymerization that will occur. In some embodiments the temperature of step a) is from 180° C. to 230° C., or 195° C. to 230° C., or 195° C. to 220° C., or from 195° C. to 210° C., or from 195° C. to 200° C.

The residence time of the mannose containing composition in the microreactor during step a) will also affect the amount or degree of polymerization that will occur. Residence time in some embodiments is from 5 to 25 seconds, or from 10 to 15 seconds.

A skilled person will understand that the degree of polymerization of in step a) can be managed, or directed, by the combination of changing the residence time in the reactor as well as modifying the temperature of the reaction. Less or more polymerization can be achieved based on the desires of the operator.

In some embodiments, before step a), the mannose is passed through a first microreactor in the presence of the acidifying catalyst at a lower temperature than the temperature of step a) described above. Said lower temperature may be from 100° C. to 180° C., or from 120° C. to 175° C., or from 140° C. to 170° C., or from 150° C. to 165° C. Some polycondensation may already take place during this step. In some embodiments the residence time of the mannose containing composition in the first microreactor is quite short, such as 5 seconds or lower, or 4 seconds or lower, or 3 seconds or lower, or 2 seconds or lower, such as from 0.5 to 2 seconds or from 0.5 to 1 second.

The first microreactor and the microreactor of step a) can have the same configuration, they may be similar, and they may even be the same microreactor, when the process is performed batch wise for example. The process of the present invention can run in batch, semi-continuous, pulse or continuous manner, preferably it is run in a continuous manner.

The manno-oligosaccharide composition from step a) is treated to neutralize the reaction catalyst. Neutralising the manno-oligosaccharide composition may be done until the composition reaches a pH of from 4 to 7. This is advantageous for an increased stability of the product, e.g. less hydrolysis over time and thus less to no change in composition of the product. Also, an advantage is that the product is then suitable for use with other ingredients that are sensitive to acids or acidic conditions. Neutralisation may be done with any suitable base. In some embodiments, in particular when the manno-oligosaccharide composition is to be used in a cosmetic or personal care product the base is caustic and/or potassium hydroxide. The neutralization can be done with 4% aqueous solution of sodium hydroxide. During this stage the neutralized solution will be diluted to a 40%-50% dry substance content.

The neutralized manno-oligosaccharide composition is then decolorized. Decolorization can be done by standard procedures known in the art. In some embodiments, the neutralized solution from step b) can be treated with hydrogen peroxide in the presence of base at 90 degrees. Caustic (4%) can be added periodically to the decolorization process to maintain the pH above 6. The reaction is monitored for color change as an endpoint. Decolorization is completed by addition of active carbon to decompose/deactivate the remaining hydrogen peroxide. The mixture is stirred with active carbon at 90 degrees C. until peroxide levels are lower than 0.5 ppm as determined by test strip.

The decolorized manno-oligosaccharide composition is then treated to remove salts by electrodialysis and or ion exchange chromotography. For example, the decolorized solution is run in a loop through a Suez WT&S Electromat™ ED pilot unit. Such electrodialysis equipment is well known in the dairy and water treatment industries. The product is treated under standard conditions for this equipment as recommended by the manufacturer until the conductivity is reduced to below 250 μS/cm. (i.e. micro-Siemems per centimeter). An approximately 90% reduction in salts can be achieved during the procedure.

The resulting product may then be further refined. Refining the produced manno-oligosaccharide composition may be done by passing it over anionic and/or cationic resins, either in a single column or multiple columns. Such refining and resins are well known in the carbohydrate arts. This is particularly advantageous when the manno-oligosaccharide composition is to be used in cosmetic applications. In some embodiments, the solution is passed through a series of columns of anionic and cationic resins at a flow rate of 2 bed volumes per hour. The skilled person would appreciate that the de-salting step can be performed in any number of known or standard ways and using standard or known resins. The objective is achieve a final product with conductivity value of less than 100 μS/cm, or less than 150 μS/cm, or less than 200 μS/cm.

The refined product of step e) can then polished by passing through a column of active carbon. In some embodiments, the solution from step e) is passed through the carbon column at a flow rate of 1 bed volume per hour.

The MOS composition of the present invention can be then isolated by removal of water through standard evaporation techniques to yield a DS of >65% preferably >70%, or between 70 and 80%.

The present invention further relates to a MOS composition obtainable by the process of the present invention. Indeed, such MOS composition has, amongst others, the improved effects as discussed herein.

The present invention will be illustrated by the following non-limiting examples.

### Example 1A: Production of MOS Composition

A mannose solution 170 L at 71% DS (C*TruSweet 016Ko from Cargill) containing 90 wt %+/−2% of mannose on a dry basis is blended with solid citric acid at a weight ratio of 100:3 on a dry basis. The resulting solution is evaporated via a thin-film evaporator up to a dry substance of 85%+/−1%. The solution is then heated to 194° C. by pumping it through a micro heat exchanger (Kreuzxtrom-reaktormodul 1694-X-19.0, KIT, IMVT) at a constant flow rate of 15 kg/hr with an overall residence time shorter than 1 seconds. The material is then pumped through a second micro heat exchanger (Kreuzxtrom-reaktormodul 2155-A-4.0, KIT, IWVT) for a period of approximately 15 seconds at a temperature of 195° C., where the main polycondensation reaction takes place.

The resulting polycondensation product (manno-oligosaccharide composition) is diluted with lye water (4% solution) to obtain a solution with 50 wt % solid content and a pH of 5+/−0.2. The solution is then cooled down to 50° C. Example 1 was run in triplicate and the results are show in the table below. The HPLC analysis (ISO 10504:1998-10) shows a manno-oligosaccharide, i.e. DP3+, content of 60 wt %.

The neutralized manno-oligosaccharide composition is then treated at 90 degrees C. with hydrogen peroxide (30 ml of 30% by weight) and 4 ml of 4% NaOH solution. The decolorization reaction is allowed to run until the solution is essentially colorless. NaOH may be added to maintain a pH of >6.

The peroxide treated manno-oligosaccharide composition was treated at 65 degrees C. with 100 g of active carbon/kg of composition (CPG® LF acid washed agglomerated coal based granular activated carbon from Chemiviron). The reaction was monitored for reduction of hydrogen peroxide with test strips from MQuant™. The reaction was considered complete when the peroxide level was determined to be below 0.5 ppm. The reaction was filtered to yield the decolorized manno-oligosaccharide composition.

The decolorized manno-oligosaccharide composition then subjected to electrodialysis to reduce the content of salts. The decolorized solution is run in a loop through a Suez WT&S Electromat™ ED pilot unit under standard conditions for this equipment as recommended by the manufacturer until the conductivity is reduced to below 250 μS/cm to yield a de-salted manno-oligosaccharide composition. Duration of the treatment was approximately 4 hours.

The de-salted manno-oligosaccharide composition was then run in a single pass at 45 degrees C. through a series of 2 160 L columns at a rate of 2 bed volumes per hour. The first column contains a cationic resin (Lewatit 52568) the second column contains an anionic resin (Lewatit S4468).

The resulting de-salted solution was then polished by passing it through a 200 L column of active carbon (CPG® LF acid washed agglomerated coal based granular activated carbon from Chemiviron) at 70 degrees C. and at a rate of 0.5 bed volumes per hour.

Finally, the highly purified manno-oligosaccharide composition was isolated by removal of excess water to yield a dry substance content of >70%. The composition had i) a Gardner value of 2.9 and ii) a ppm content of furfural and hydroxymethyl-furfural below detection limit and a Gardner value of 2.8.

TABLE 1

| | | | | | | MW | MW |
|---|---|---|---|---|---|---|---|
| | | Free | Free | | | (number | (weight |
| | DS | mannnose | glucose | DP2 | DPn | aver- | aver- |
| Example 1 | (%) | (%) | (%) | (%) | (%) | age) | age) |
| 1A | 72 | 18 | 1 | 18 | 60 | 403 | 886 |
| 1B | 66 | 18 | 1 | 18 | 60 | 402 | 964 |
| 1C | 72 | 13.9 | 3.2 | 18.7 | 60.2 | 377 | 686 |

### Evaluation of MOS in Skin Assay

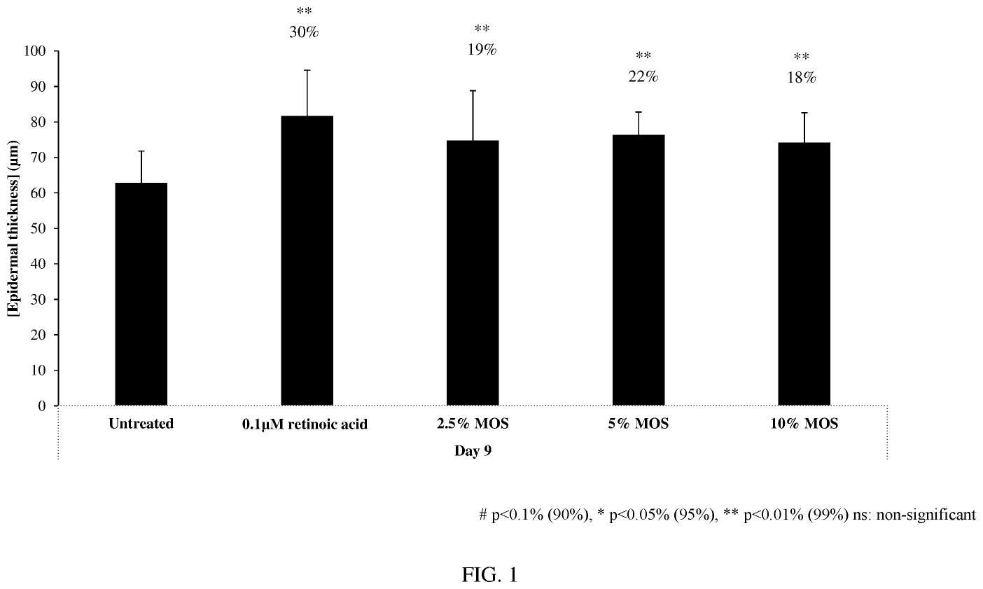

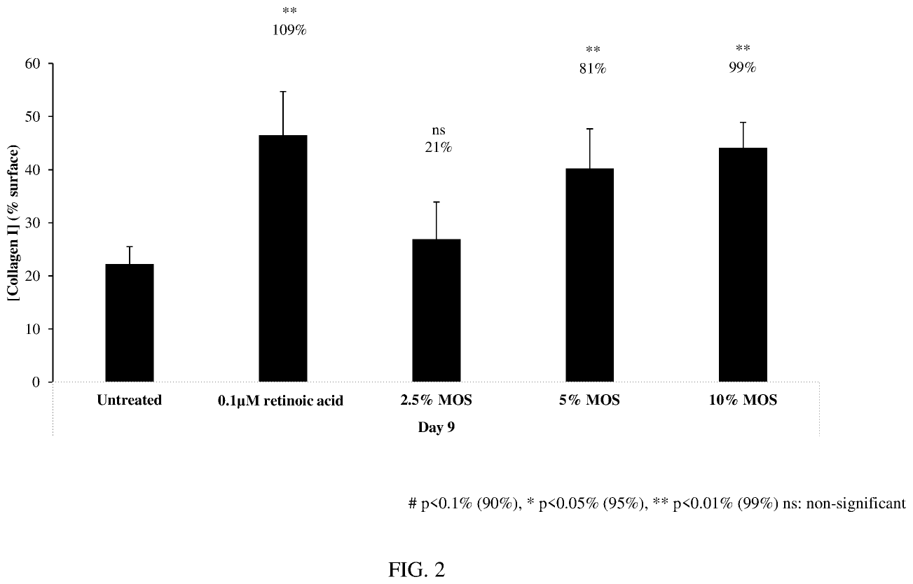

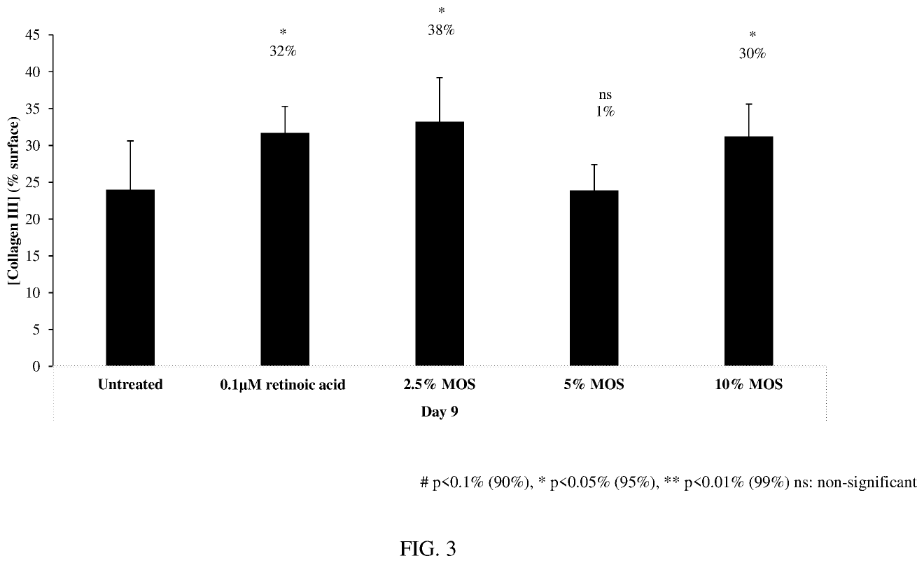

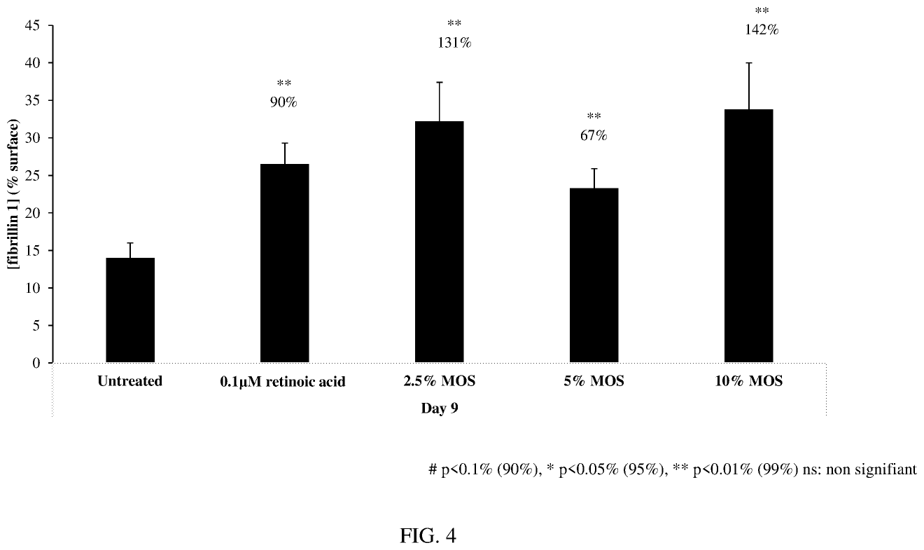

Evaluation in the skin assay was performed by a third-party testing firm Laboratoire BIO-EC 1, Chemin de Saulxier 91160 Longjumeau FRANCE. Skin samples from 68-year old Caucasian woman were harvested and put on a BEM culture medium maintained in 37 degree C., humid, 5% CO2 atmosphere. MOS product from Example 1 and retinoic acid control were applied on days 0, 1, 2, 5, and 7. The cell cultures were evaluated on day 9 by microscope and results are summarized in FIGS. 1-4.

The assay results demonstrated significant and surprising improvement in epidermal thickness, collagen I, collagen III, and Fibrillin I relative to the positive control.

1. A purified composition comprising a mannose oligosaccharide where the mannose oligosaccharide has a DPn of >50%, a dry substance content of mannose oligosaccharide of >65%, and a Gardner color value of less than 5.0.

2. The composition of claim 1 having a connectivity value of a 50% by weight solution in water of <500 microsimons/cm.

3. The composition of claim 1 having a mannose oligosaccharide dry substance content of between 65% and 80%.

4. The composition of claim 3 having a Gardner color value of between 1.0 and 5.0.

5. (canceled)

6. A method of using a composition of claim 1 to improve the health or thickness of the skin of a mammal comprising applying an effective amount of a composition of claim 1 to the skin of a mammal.

7.-12. (canceled)

13. A personal care or cosmetic product comprising a purified mannose oligosaccharide composition wherein the purified mannose oligosaccharide has a DPn of >50%, a dry substance content of mannose oligosaccharide of >65%, and a Gardner color value of less than 5.0.

14. The product of claim 6 comprising a purified mannose oligosaccharide composition having a connectivity value of a 50% by weight solution in water of <500 microsimons/cm.

15. The product of claim 6 comprising a purified mannose oligosaccharide composition having a mannose oligosaccharide dry substance between 65% and 80%.

16. The product of claim 15 comprising a purified mannose oligosaccharide composition having a Gardner color value of between 1.0 and 5.0.

17. The composition of claim 13, that is a cosmetic, skin cream, lotion, sunscreen, skin cleanser, or skin conditioner.

18. The composition of claim 16 that is a cosmetic, skin cream, lotion, sunscreen, skin cleanser, or skin conditioner.
